(19) United States
(12) Patent Application Publication
(10) Pub. No.: US 2020/0139145 A1
(43) Pub. Date: May 7, 2020

(54) HIGH FREQUENCY ORAL THERAPEUTIC DEVICE

(71) Applicant: YOZMA BMTECH CO., LTD., Seongnam-si Gyeonggi-do (KR)

(72) Inventor: Seung Moo HAN, Yongin-si Gyeonggi-do (KR)

(73) Assignee: YOZMA BMTECH CO., LTD., Seongnam-si Gyeonggi-do (KR)

(21) Appl. No.: 16/631,564

(22) PCT Filed: Apr. 24, 2018
(86) PCT No.: PCT/KR2018/004719

§ 371 Date: Jan. 16, 2020

(30) Foreign Application Priority Data
Jul. 21, 2017 (KR) 10-2017-0092826

(51) Int. Cl. A61N 1/40; A61N 1/08; A61N 1/02

(52) U.S. Cl. CPC A61N 1/403; A61N 1/06; A61N 1/025; A61N 1/08

(57) ABSTRACT

The present invention relates to a radiofrequency oral therapeutic device, and provides a radiofrequency oral therapeutic device including: a radiofrequency generator configured to generate a radiofrequency current; an adaptor connected to the radiofrequency generator via a cable; a probe configured to be detachably coupled to the adaptor and to apply the radiofrequency current, transferred from the radiofrequency generator through the adaptor, to an intraoral skin of a user; and a control unit configured to control the radiofrequency generator; wherein the probe is connected to the control unit through the adaptor and transmits a probe identifier (ID), and the control unit controls the radiofrequency generator based on the probe ID.

### TECHNICAL FIELD

The present invention relates to a high frequency therapeutic device, and more specifically to a high frequency therapeutic device that can be used for the intraoral skin.

### BACKGROUND ART

A radiofrequency (high frequency) therapeutic device is a device that promotes the physiological action of the skin through the generation of collagen in the skin, the removal of pigments, etc. by using the principle that when a radiofrequency current of 300 to 5000 kHz is applied to an affected part, thermal energy is generated while the radiofrequency current is passing through the skin, which is a resistor, thereby allowing the effects of tightening and whitening the skin to be expected.

Such a radiofrequency therapeutic device employs a method of heating an affected part by directly applying a radiofrequency current to the face of a user. Although a therapeutic effect increases in proportion to temperature, a bad side effect, such as a facial burn, a hot flush, or the like, may occur, and thus a problem arises in that temperature cannot be sufficiently increased. Accordingly, the conventional high frequency therapeutic device is used with the facial portion thereof limited to a temperature of 40 to 45° C.

Meanwhile, when such a radiofrequency therapeutic device is used for the intraoral skin, a generated wound or scar is not exposed to the outside even when heating is performed to a higher temperature than that in the case where radiofrequency therapeutic device is used for the facial skin, and thus a therapeutic effect can be increased by increasing heating temperature. In particular, although the part between the cheekbone and a corner of the mouth is an area from which an effect cannot be obtained by using the conventional method. However, when an intraoral stimulation method is used for the part, an advantage arises in that the skin of the corresponding part can be effectively smoothed. However, the conventional high frequency therapeutic device has been proposed for the purpose of being used only for the external skin, such as the face, but a technology that enables a radiofrequency therapeutic device to be used inside the oral cavity has not been particularly proposed yet.

### PRIOR ART DOCUMENT

Korean Patent No. 10-1635596 (published on Jul. 4, 2016)

### DISCLOSURE

### Technical Problem

The present invention has been conceived to overcome the above-described limitations, and an object of the present invention is to provide a radiofrequency therapeutic device that can be used in the oral cavity.

Another object of the present invention is to provide a radiofrequency therapeutic device that can easily determine whether or not a probe has been used by using a probe ID, automatically determine a therapeutic mode based on the probe ID, and generate a radiofrequency current based on the therapeutic mode.

Another object of the present invention is to provide a radiofrequency therapeutic device that allows a probe to be detachably coupled using an adaptor, thereby being able to be used in internal body parts other than the oral cavity, e.g., the vagina, only by changing probes.

### Technical Solution

In order to accomplish the above objects, the present invention provides a radiofrequency oral therapeutic device including: a radiofrequency generator configured to generate a radiofrequency current; an adaptor connected to the radiofrequency generator via a cable; a probe configured to be detachably coupled to the adaptor and to apply the radiofrequency current, transferred from the radiofrequency generator through the adaptor, to an intraoral skin of a user; and a control unit configured to control the radiofrequency generator; wherein the probe is connected to the control unit through the adaptor and transmits a probe identifier (ID), and the control unit controls the radiofrequency generator based on the probe ID.

In this case, the probe preferably includes an inclined portion that is formed at a location ½ to ⅔ of an overall length of the probe away from the adaptor in a longitudinal direction of the probe so that the probe can be easily operated when it is inserted through a mouth of the user.

The probe further may further include: a metal plate configured to come into direct contact with the intraoral skin of the user and to apply the radiofrequency current; a temperature sensor configured to measure the temperature of the metal plate; and a microprocessor configured to store the probe ID and to transmit the probe ID to the control unit through the adaptor when power is applied.

The control unit may automatically change a therapeutic mode based on the probe ID transmitted from the microprocessor.

The microprocessor may further include memory configured to record information, indicating that the probe has been used, when the radiofrequency current is applied from the radiofrequency generator; and, when power is applied, the microprocessor, after transmitting the probe ID, checks the memory and transmits a signal indicating whether or not the probe has been used to the control unit.

The therapeutic mode is preferably configured such that at least any one of frequency, a pulse shape, and temperature is set therein.

The control unit may transmit a control signal corresponding to the therapeutic mode to the radiofrequency generator, and the radiofrequency generator may generate a radiofrequency current corresponding to the control signal and transmit the radiofrequency current to the probe through the adaptor.

The microprocessor of the probe may transmit the measured temperature of the metal plate to the control unit at predetermined periods while measuring the temperature by using the temperature sensor, and the control unit may compare the received temperature with a preset threshold value and transmit a control signal adapted to stop the operation of the radiofrequency generator when the received temperature exceeds the preset threshold value.

According to another aspect of the present invention, there is provided a radiofrequency oral therapeutic device including: a radiofrequency generator configured to generate a radiofrequency current; an adaptor connected to the radiofrequency generator via a cable; an oral probe configured to be detachably coupled to the adaptor and to apply the radiofrequency current, transferred from the radiofrequency generator through the adaptor, to an intraoral skin of a user; a vaginal probe configured to be detachably coupled to the adaptor and to apply the radiofrequency current, transferred from the radiofrequency generator through the adaptor, to an intravaginal skin of a user; and a control unit configured to control the radiofrequency generator; wherein the adaptor is detachably coupled to any one of the oral probe and the vaginal probe, the oral probe or vaginal probe is connected to the control unit through the adaptor and transmits a probe identifier (ID), and the control unit controls the radiofrequency generator based on the probe ID.

### Advantageous Effects

According to the present invention, there may be provided the radiofrequency therapeutic device that can be used in the oral cavity.

The present invention may provide the radiofrequency therapeutic device that can easily determine whether or not a probe has been used by using a probe ID, automatically determine a therapeutic mode based on the probe ID, and generate a radiofrequency current based on the therapeutic mode.

The present invention may provide the radiofrequency therapeutic device that allows a probe to be detachably coupled using an adaptor, thereby being able to be used in internal body parts other than the oral cavity, e.g., the vagina, only by changing probes.

### DESCRIPTION OF DRAWINGS

FIG. 1 is a diagram showing the overall configuration of a radiofrequency oral therapeutic device (100) according to an embodiment of the present invention;

FIGS. 2 and 3 are views showing the appearance of a probe (30);

FIG. 4 is a diagram showing the internal configuration of the probe (40);

FIG. 5 is a flowchart illustrating the overall operation of the radiofrequency oral therapeutic device (100) described with reference to FIGS. 1 to 4;

FIG. 6 is a diagram showing a radiofrequency oral therapeutic device (100) according to another embodiment of the present invention; and

FIG. 7 is a view showing the appearance of a vaginal probe (30-1) used in the embodiment of FIG. 6.

### BEST MODE

Embodiments of the present invention will be described in detail below with reference to the accompanying drawings.

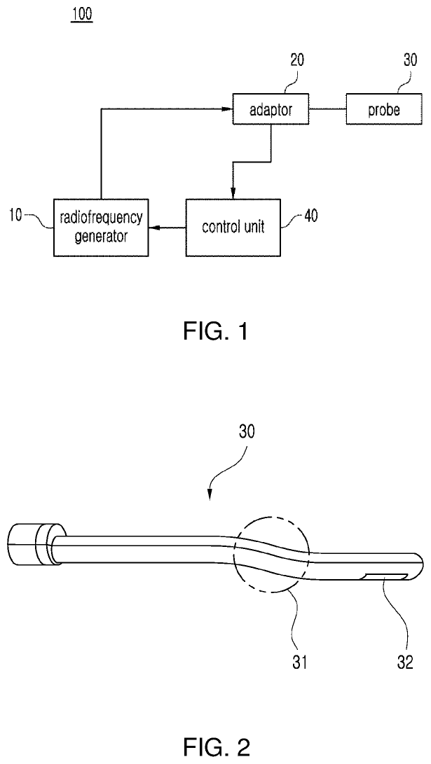

FIG. 1 is a diagram showing the overall configuration of a radiofrequency oral therapeutic device 100 according to an embodiment of the present invention.

Referring to FIG. 1, the radiofrequency oral therapeutic device 100 according to the present embodiment includes a radiofrequency generator 10, an adaptor 20, a probe 30, and a control unit 40.

The radiofrequency generator 10 functions to generate a radiofrequency current. The radiofrequency generator 10 generates a radiofrequency current in response to a control signal from the control unit 20, and transfers the radiofrequency current to the adaptor 20 via a cable. Since the radiofrequency generator 10 itself is known by the prior art, a detailed description thereof will be omitted here.

The adaptor 20 is connected to the radiofrequency generator 10 by the cable, is detachably coupled to the probe 30 to be described below, and applies the radiofrequency current, transferred from the radiofrequency generator 10, to the probe 30. Furthermore, as described later, the adaptor 20 functions to transfer a probe identifier (ID), transferred from the probe 30, to the control unit 40.

The probe 30 is detachably coupled to the adaptor 20, and functions to apply the radiofrequency current, transferred from the radiofrequency generator 10 through the adaptor 20, to the intraoral skin of a user.

Furthermore, the probe 30 is connected to the control unit 40 through the adaptor 20, and functions to transmit the probe ID to the control unit 40 through the adaptor 20.

The control unit 40 functions to control the overall operation of the radiofrequency generator 10. The control unit 40 controls the radiofrequency generator 10 based on the probe ID transferred from the probe 30.

Next, the probe 40 will be described with reference to FIGS. 2 to 4.

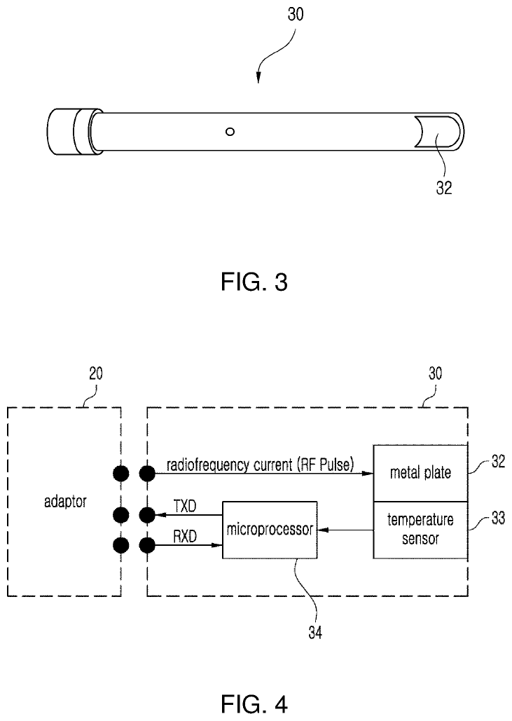

FIGS. 2 and 3 are views showing the appearance of the probe 30, and FIG. 4 is a diagram showing the internal configuration of the probe 40.

Referring to FIGS. 2 to 4, the probe 40 has a shape extending in a rectilinear line. A metal plate 32 that comes into contact with the intraoral skin and applies a radiofrequency current is formed at one end of the probe 40. The metal plate 32 is connected to the adaptor 30 through the inside of the probe 40, and applies a radiofrequency current, transferred from the adaptor 30, to the intraoral skin of a user.

Meanwhile, the radiofrequency oral therapeutic device 100 according to the present invention is to be used in contact with the intraoral skin of a user, and is thus inserted into an oral cavity through the mouth of the user. Accordingly, it is preferred that an inclined portion 31 that is inclined at a predetermined angle, e.g., within the range of 5 to 10 degrees, is formed at a location about ½ to ⅔ of the overall length away from the adaptor 20, preferably a location about ⅔ of the overall length away from the adaptor 20, in the longitudinal direction of the probe 40 so that the probe 30 can be operated through the mouth.

Meanwhile, as shown in FIG. 4, the probe 30 further includes a metal plate 32, a temperature sensor 33, and a microprocessor 34.

The metal plate 32 is a means that comes into direct contact with the intraoral skin of a user and applies radiofrequency current. The temperature sensor 33 is a sensor that measures the temperature of the metal plate 32.

The microprocessor 34 stores a probe ID in internal memory, and functions to transmit the probe ID to the control unit 20 through the adaptor 20 when power is applied.

In the present invention, the probe 30 must be disposable because it is used inside the oral cavity, and a probe ID is uniquely allocated to each probe 30 separately.

Furthermore, the microprocessor 34 contains nonvolatile memory, and records information indicating that the probe 30 has been used in the memory when a radiofrequency current is applied from the radiofrequency generator 10 (i.e., when the probe 30 is used). Whether or not the probe 30 is reused is determined based on the memory.

Accordingly, when power is applied, the microprocessor 34, after transmitting the probe ID, checks the memory, and transmits a signal indicating whether or not the probe 30 has been used to the control unit 40. In this case, it is preferable that the control unit 40 allows a subsequent operation only when the probe 30 has not been used and outputs a corresponding message through a display unit and stops an operation when the probe 30 has been used.

Furthermore, the microprocessor 34 transmits the temperature, measured by the temperature sensor 33, to the control unit 40.

The control unit 40 determines that a signal indicating that the probe 30 has not been used has been received, and automatically changes a therapeutic mode based on the probe ID transmitted from the microprocessor 34 of the probe 30. In this case, the therapeutic mode refers to a preset at least one mode in which various types of variables for the operation of the radiofrequency therapeutic device 100, such as frequency, a pulse shape, and temperature, are preset.

The control unit 40 sets a therapeutic mode based on the probe ID, and transmits a control signal corresponding to the therapeutic mode to the radiofrequency generator 10. This control signal contains information for the setting of parameters, such as frequency, a pulse shape, and temperature.

The radiofrequency generator 10 generates a radiofrequency current corresponding to the control signal from the control unit 40, and transmits the generated radiofrequency current to the probe 30 through the adapter 20. While radiofrequency therapeutic device 100 is operating, the microprocessor 34 of the probe 30 transmits measured temperature to the controller 40 at regular intervals while continuously measuring the temperature of the probe 30 by using the temperature sensor 33. The controller 40 compares the received temperature with a preset threshold value, and transmits a control signal for the stopping of the operation of the radiofrequency generator 10 when the received temperature exceeds the preset threshold value threshold. Then the radiofrequency generator 10 stops its operation.

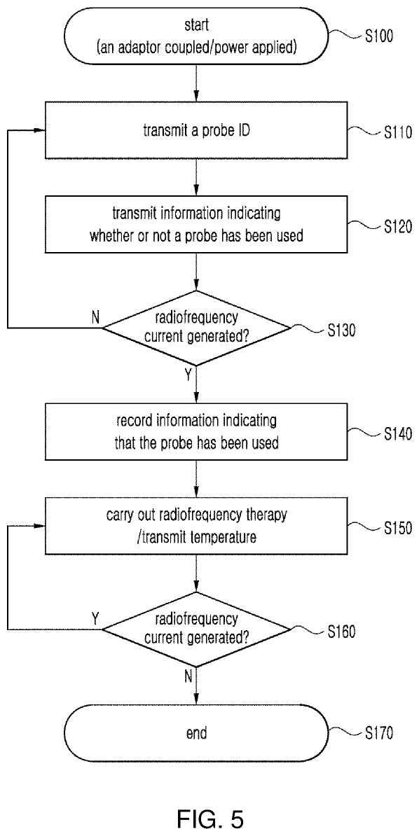

FIG. 5 is a flowchart illustrating the overall operation of the radiofrequency oral therapeutic device 100 described with reference to FIGS. 1 to 4.

Referring to FIG. 5, the probe 30 and the adaptor 20 are coupled to each other, and, when power is applied, the operation of the radiofrequency oral therapeutic device 100 starts at step S100.

First, the microprocessor 34 of the probe 30 transmits a probe ID, stored in the memory, to the control unit 40 through the adaptor 20 at step S110.

Furthermore, the microprocessor 34 transmits a signal indicating whether the probe 30 has been used, stored in the memory, to the control unit 40 at step S120.

When a signal indicating that the probe 30 has not been used is received, the control unit 40 automatically changes the therapeutic mode, and transmits a control signal corresponding to the corresponding therapeutic mode to the radiofrequency generator 10 so that the radiofrequency generator 10 generates a radiofrequency current at step S130.

The radiofrequency generator 10 generates the radiofrequency current and transfers the radiofrequency current to the probe 30 through the adaptor 20, and the microprocessor 34 of the probe 30 records information indicating that the probe 30 has been used in the memory at step S140.

The metal plate 32 of the probe 30 applies a radiofrequency current to the intraoral skin of a user, and an operator carries out radiofrequency therapy for the user while operating the probe 30. While this operation is being carried out, the temperature sensor 33 of the probe 30 measures the temperature of the metal plate 32 at preset periods and the microprocessor 34 transmits the temperature, measured by the temperature sensor 33, to the control unit 40 at step S150.

The control unit 40 compares the temperature transmitted from the probe 30 with a preset threshold value, and transmits a control signal adapted to stop the generation of radiofrequency current to the radiofrequency generator 10 when the temperature exceeds the preset threshold value, thereby stopping operation, and continues to carry out radiofrequency therapy when the temperature does not exceed the preset threshold value at steps S160 and S170.

The control unit 40 determines both whether to carry out radiofrequency therapy based on temperature and whether the end time based on the preset therapeutic mode has been reached, and may stop or continue the operation accordingly. Furthermore, the control unit 40 may transmit a control signal to the radiofrequency generator 10 in order to change the pulse shape, frequency, etc. of the radiofrequency current over time according to the therapeutic mode.

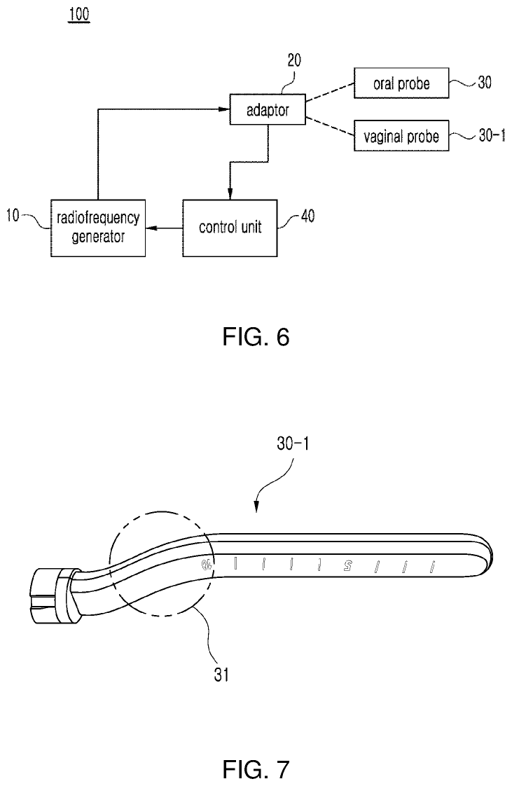

FIG. 6 is a diagram showing a radiofrequency oral therapeutic device 100 according to another embodiment of the present invention.

The radiofrequency oral therapeutic device 100 according to the embodiment of FIG. 6 is the same as the embodiment of FIGS. 1 to 5, and is different from the embodiment of FIGS. 1 to 5 in that a vaginal probe 30-1 is further included and an oral probe 30 and the vaginal probe 30-1 are coupled to an adaptor 20 according to therapeutic target sites.

In the embodiment of FIG. 6, when the probe ID is received from the probe 30, the control unit 40 operates to determine whether the corresponding probe is an oral probe or a vaginal probe based on the probe ID and to determine a corresponding therapeutic mode. For this purpose, the probe ID includes information indicating that the corresponding probe is an oral probe or a vaginal probe.

FIG. 7 is a view showing the appearance of the vaginal probe 30-1 used in the embodiment of FIG. 6.

When the vaginal probe 30-1 shown in FIG. 7 is compared with the oral probe 30 of FIGS. 2 and 3, the vaginal probe 30-1 is different from the oral probe 30 in that an inclined portion 31 is formed over a larger length at a location closer to an adaptor 20 in the longitudinal direction of the probe 30.

According to the embodiment of FIGS. 6 and 7, an advantage arises in that the oral probe and the vaginal probe can be selectively coupled to and used with the adaptor 20 in the single radiofrequency therapeutic device 100.

Although the present invention has been described with reference to the preferred embodiments of the present invention, the present invention is not limited to the embodiments, and it will be apparent that other various modifications and alterations may be made.

1. A radiofrequency oral therapeutic device comprising:
a radiofrequency generator configured to generate a radiofrequency current;
an adaptor connected to the radiofrequency generator via a cable;
a probe configured to be detachably coupled to the adaptor and to apply the radiofrequency current, transferred from the radiofrequency generator through the adaptor, to an intraoral skin of a user; and
a control unit configured to control the radiofrequency generator;
wherein the probe is connected to the control unit through the adaptor and transmits a probe identifier (ID), and the control unit controls the radiofrequency generator based on the probe ID.

2. The radiofrequency oral therapeutic device of claim 1, wherein the probe comprises an inclined portion that is formed at a location ½ to ⅔ of an overall length of the probe away from the adaptor in a longitudinal direction of the probe so that the probe can be easily operated when it is inserted through a mouth of the user.

3. The radiofrequency oral therapeutic device of claim 2, wherein the probe further comprises:
a metal plate configured to come into direct contact with the intraoral skin of the user and to apply the radiofrequency current;
a temperature sensor configured to measure temperature of the metal plate; and
a microprocessor configured to store the probe ID and to transmit the probe ID to the control unit through the adaptor when power is applied.

4. The radiofrequency oral therapeutic device of claim 3, wherein the control unit automatically changes a therapeutic mode based on the probe ID transmitted from the microprocessor.

5. The radiofrequency oral therapeutic device of claim 3, wherein:
the microprocessor further comprises memory configured to record information, indicating that the probe has been used, when the radiofrequency current is applied from the radiofrequency generator; and
when power is applied, the microprocessor, after transmitting the probe ID, checks the memory and transmits a signal indicating whether or not the probe has been used to the control unit.

6. The radiofrequency oral therapeutic device of claim 4, wherein the therapeutic mode is configured such that at least any one of frequency, a pulse shape, and temperature is set therein.

7. The radiofrequency oral therapeutic device of claim 4, wherein the control unit transmits a control signal corresponding to the therapeutic mode to the radiofrequency generator, and the radiofrequency generator generates a radiofrequency current corresponding to the control signal and transmits the radiofrequency current to the probe through the adaptor.

8. The radiofrequency oral therapeutic device of claim 7, wherein the microprocessor of the probe transmits the measured temperature of the metal plate to the control unit at predetermined periods while measuring the temperature by using the temperature sensor, and the control unit compares the received temperature with a preset threshold value and transmits a control signal adapted to stop operation of the radiofrequency generator when the received temperature exceeds the preset threshold value.

9. A radiofrequency oral therapeutic device comprising:
a radiofrequency generator configured to generate a radiofrequency current;
an adaptor connected to the radiofrequency generator via a cable;
an oral probe configured to be detachably coupled to the adaptor and to apply the radiofrequency current, transferred from the radiofrequency generator through the adaptor, to an intraoral skin of a user;
a vaginal probe configured to be detachably coupled to the adaptor and to apply the radiofrequency current, transferred from the radiofrequency generator through the adaptor, to an intravaginal skin of a user; and
a control unit configured to control the radiofrequency generator;
wherein the adaptor is detachably coupled to any one of the oral probe and the vaginal probe, the oral probe or vaginal probe is connected to the control unit through the adaptor and transmits a probe identifier (ID), and the control unit controls the radiofrequency generator based on the probe ID.
